(19) United States
(12) Patent Application Publication
(10) Pub. No.: US 2025/0235484 A1
(43) Pub. Date: Jul. 24, 2025

(54) METHODS FOR MAKING EXTRACELLULAR VESICLES, AND COMPOSITIONS AND METHODS OF USE THEREOF

(71) Applicant: EVIA LIFE SCIENCES INC., Henderson, NV (US)

(72) Inventor: Takahiro Ochiya, Tokyo (JP)

(21) Appl. No.: 18/703,686

(22) PCT Filed: Oct. 24, 2022
(86) PCT No.: PCT/IB2022/000656

§ 371 Date: Apr. 22, 2024

Related U.S. Application Data
(60) Provisional application No. 63/270,875, filed on Oct. 22, 2021.
(60) Provisional application No. 63/333,854, filed on Apr. 22, 2022.

(51) Int. Cl. A61K 35/36; A61P 17/00; C12N 5/071

(52) U.S. Cl. CPC A61K 35/36; A61P 17/00; C12N 5/0629; C12N 2501/999

(57) ABSTRACT

Methods of making extracellular vesicles (EVs) by culturing keratinocytes in culture media including a ROCK inhibitor, and harvesting EVs secreted by the keratinocytes are provided. In some embodiments, the EVs include or consist of exosomes. Typically, the proliferation of the keratinocytes and/or secretion of EVs is increased in the presence of the ROCK inhibitor compared to is absence. EVs made according to the disclosed methods, and pharmaceutical compositions formed therefrom are also provided. The pharmaceutical compositions can include an effective amount of the EVs to, for example, serve a nutraceutical and therapeutic application such as improving skin, treating a skin-related disease or disorder, or enhancing recovering from injury.

### CROSS-REFERENCE TO RELATED APPLICATION

This application claims the benefit of and priority to U.S. Ser. No. 63/270,875 filed Oct. 22, 2021, and U.S. Ser. No. 63/333,854 filed Apr. 22, 2022, which are specifically incorporated by reference herein in their entireties.

### FIELD OF THE INVENTION

The field of the invention generally is related to compositions and methods of culturing cells, collection of extracellular vesicles, and compositions and methods of use thereof.

### BACKGROUND OF THE INVENTION

Extracellular vesicles (EVs) are secretory lipid membranes with the ability to regulate cellular functions by exchanging biological components between different cells (Nasiri, et al., Stem Cell Research & Therapy volume 11, Article number: 421 (2020)). Skin cells such as keratinocytes, fibroblasts, melanocytes, and inflammatory cells can secrete different types of EVs depending on their biological state. These vesicles can influence the physiological properties and pathological processes of skin, such as pigmentation, cutaneous immunity, and wound healing. Since keratinocytes constitute the majority of skin cells, secreted EVs from these cells may alter the pathophysiological behavior of other skin cells.

The nature of EVs as biological carriers has potential in different skin therapy purposes including repair, regeneration, and rejuvenation (Basu, et al., Expert Opin Biol Ther. 16 (4): 489-506 (2016)). The immediate deterioration of primary human keratinocytes during culture limits their utility in drug discovery studies as well as a source for materials for nutraceutical and therapeutic use regenerative medicine. To make these therapeutic approaches accessible to patients, good manufacturing practice (GMP) as standard protocols are needed to ensure the quality of EVs used (Chen, et al., Tzu-Chi Med J., 32 (2): 113 (2019), Lener, et al., J Extracellular Vesicles, 4 (1): 30087 (2015)).

Thus, it is object of the invention to provide improved methods culturing keratinocytes and making and harvesting EVs therefrom, compositions including the EVs, and methods of use thereof.

### SUMMARY OF THE INVENTION

Methods of making extracellular vesicles (EVs) by culturing keratinocytes in culture media including a ROCK inhibitor, and harvesting EVs secreted by the keratinocytes are provided. In some embodiments, the EVs include or consist of exosomes.

Typically, the proliferation of the keratinocytes and/or secretion of EVs is increased in the presence of the ROCK inhibitor compared to in its absence. An exemplary ROCK inhibitor is Y-27632. In some embodiments, the cells are also cultured with an inhibitor of TGFβ signaling. An exemplary inhibitor of TGFβ signaling is A83-01.

In preferred embodiments, the keratinocytes are primary keratinocytes.

EVs made according to the disclosed methods, and pharmaceutical compositions formed therefrom, are also provided. The pharmaceutical compositions can include an effective amount of the EVs to, for example, serve a nutraceutical and therapeutic application such as improving skin, treating a skin-related disease or disorder, or enhancing recovering from injury, and such therapeutic and non-therapeutic methods and uses are also provided. In some embodiments, the compositions are used to treat or prevent dryness, irritation, stress, allergies, infection and/or heat/sweating of the skin. For example, in some embodiments, the methods include administering EV's or a composition thereof to a subject in need thereof to treat or prevent atopic dermatitis. In some embodiments, a composition is administered in an effective amount to reduce or prevent one or more symptoms and/or biological or physiological indicators of atopic dermatitis.

In some embodiments, the exosomes can increase the expression of Type I collagen (COL1A1) and/or elastin; reduce expression of thymic stromal lymphopoietin (TSLP), Th2, eosinophil-recruiting chemokines, inflammatory cytokines such as IL-33 and IL-25, or any combination thereof in cells to which they are contacted.

### BRIEF DESCRIPTION OF THE DRAWINGS

FIGS. 1A-1H are 4× (FIGS. 1A, 1C, 1E, 1G) and 10× (FIGS. 1B, 1D, 1F, 1H) micrographs showing keratinocytes cultured in keratinocyte media alone on day 2 (FIGS. 1A, 1B), day 3 (FIGS. 1C, 1D), day 7 (FIGS. 1E, and 1F), and following the addition of Y-27632 on day 9 (FIGS. 1G and 1H).

FIGS. 2A-2F are 4× (FIGS. 2A, 2C, 2E) and 10× (FIGS. 2B, 2D, 2F) micrographs showing keratinocytes cultured in keratinocyte alone (FIGS. 2A, 2B), with Y-27632 (FIGS. 2C, 2D), and with Y-27632+A83-01 (FIGS. 2E, 2F) on day 17.

FIGS. 3A-3F are 4× (FIGS. 3A, 3C, 3E) and 10× (FIGS. 3B, 3D, 3F) micrographs showing keratinocytes cultured in keratinocyte alone (FIGS. 3A, 3B), with Y (FIGS. 3C, 3D), and with Y-27632+A83-01 (FIGS. 3E, 3F) on day 20.

FIG. 4 is a bar graph showing the concentration of extracellular vesicle (EV) particles (×10\hat{ }9 particles/ml) in medium only, control keratinocytes, keratinocytes cultured with Y-27632 (Y), and keratinocytes cultured with Y-27632+A83-01 (A).

FIG. 5 is a bar graph showing the particle weight (ratio) of control keratinocytes, keratinocytes cultured with Y-27632 (Y), and keratinocytes cultured with Y-27632+A83-01 (A).

FIG. 6 is a bar graph showing gene expression level of Type I collagen (COL1A1) and elastin in human fibroblast cells 72 hours after the addition of 1,000 exosomes per cell. Relative expression level (genes/actin): The expression level of each gene was calculated using the amount of beta-actin as a control.

FIG. 7A is a flow chart of an experimental protocol for analysis of the effect of extracellular vesicles (EVs) secreted from cultured keratinocytes on the gene expression in an in vitro model for atopic dermatitis. FIG. 7B is a bar graph showing gene expression of TSLP, IL-25, and IL-33 in untreated control epidermal keratinocytes compared to epidermal keratinocytes treated with EV's prepared by normal keratinocyte culture or long-term keratinocyte culturing.

### DETAILED DESCRIPTION OF THE INVENTION

### I. Definitions

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients.

As used herein, the term “pharmaceutically acceptable carrier” encompasses any of the standard pharmaceutical carriers, such as a phosphate buffered saline solution, water and emulsions such as an oil/water or water/oil emulsion, and various types of wetting agents.

As used herein, the terms “effective amount” or “therapeutically effective amount” means a dosage sufficient to alleviate one or more symptoms of a disorder, disease, or condition being treated, or to otherwise provide a desired pharmacologic and/or physiologic effect. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “prevention” or “preventing” means to administer a composition to a subject or a system at risk for or having a predisposition for one or more symptom caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

As used herein, “substantially changed” means a change of at least e.g., 5%, 10%, 15%, 20%, 25%, 30%, 35%, 40%, 45%, 50%, 75%, 100%, or more relative to a control.

As used herein, the term “purified,” “isolated,” and like terms relate to the isolation of a molecule or compound in a form that is substantially free (at least 60% free, preferably 75% free, and most preferably 90% free) from other components normally associated with the molecule or compound in a native environment.

As used herein, the term “antibody” refers to natural or synthetic antibodies that bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that bind the target antigen.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “inhibit” or “reduce” means to decrease, hinder or restrain a particular characteristic such as an activity, response, condition, disease, or other biological parameter. It is understood that this is typically in relation to some standard or expected value, i.e., it is relative, but that it is not always necessary for the standard or relative value to be referred to. “Inhibits” or “reduce” can also mean to hinder or restrain the synthesis, expression or function of a protein relative to a standard or control. This can include, but is not limited to, the complete ablation of the activity, response, condition, or disease. Inhibition may also include, for example, a 10% reduction in the activity, response, condition, disease, or other biological parameter as compared to the native or control level. Thus, the reduction can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any amount of reduction in between as compared to native or control levels.

As used herein, “primary cell” refers to a non-immortalized cell taken from a living organism or tissue source.

As used herein, “prolonging viability” of a cell, such as a primary cell, refers to extending the duration of time the cell is capable of normal growth and/or survival.

As used herein, “senescence” refers to the point at which a cell is no longer capable of undergoing mitosis (cell division).

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed. Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. Methods of Culturing Keratinocytes

Keratinocytes are cells that are found in the epidermis that produces keratin. Keratinocytes make up about 90% of epidermal cells. Keratinocytes are produced by keratinocyte stem cells in the basal layer of the epidermis. It has been discovered that small molecule signaling inhibitors are useful for maintaining various regenerative functions of primary human keratinocytes, including growth factor productivity which induces regeneration of skin epithelia, reconstitution of a functional epidermal barrier, and production of extracellular vesicles for skin regeneration. Importantly, these culture conditions allow primary human keratinocytes to retain production of extracellular vesicles. Thus, disclosed are methods of making, harvesting, and using EVs from long term cultured keratinocytes. Such EVs can be used in a variety of applications including, but not limited to nutraceutical and therapeutic interventions such as cell-free skin regeneration and/or disease treatment, and research-based platforms to facilitate keratinocyte-based drug development for treatment of the same. Cells obtained according to the disclosed culturing methods are also expressly provided, as are pharmaceutical compositions thereof, and therapeutic and non-therapeutic methods of use thereof, e.g., for the treatment of skin disease and conditions as described in more detail elsewhere herein with respect to EVs.

The results in the experiments below show that Y-27632 (Rock inhibitor) alone, and a combination of Y-27632 and A83-01 (TGFβ signaling inhibitor), can enhance long-term culturing of keratinocytes and increases accumulation of EVs. Thus, the disclosed culturing methods typically include a ROCK inhibitor and optionally one or more additional small molecules, including, but not limited to a TGFβ inhibitor. In some embodiments, a TGFβ signaling inhibitor is not included.

The keratinocytes are typically cultured in a tissue culture media including a ROCK inhibitor and optionally one or more additional small molecules. The tissue culture media can be a sterile, liquid medium for the long-term, serum-free culture of human epidermal keratinocytes such as EpiLife™ Medium, though any suitable media for culturing keratinocytes can be used, such as Keratinocyte Growth Medium 2 (Ready-to-use) (Promocell; C20011), HuMedia-KG2 (KK-2150S) (Kurabou), and KGM-Gold™ Keratinocyte Growth Medium BulletKit™ (LONZA).

The keratinocytes are cultured in the presence of the inhibitor and/or other inhibitor(s) for a period of time sufficient to increase proliferation of the cells and/or increase the number of extracellular vesicles that can be collected relative to untreated cells. In some embodiments, the keratinocytes are cultured in the presence of the inhibitor(s) for at least 1 day, 2 days, 3 days, 4 days, 5 days, 6 days, 7 days, 8 days, 9 days, 10 days, 11 days, 12 days, 13 days, 14 days, 15 days, at least 20 days, at least 40 days, at least 60 days, at least 100 days, at least 150 days, at least 200 days, at least 250 days, at least 300 days, at least 350 days, at least 400 days, at least 450 days, or at least 500 days. Typically, the cells are cultured with the inhibitor(s) for 14 days or more.

In some embodiments, the cells are cultured for some period of time without the inhibitor(s) (e.g., 1 days, 2 days, 3 days, 4 days, 5 days, 6 days, 7 days, 8 days, 9 days, 10 days, 11 days, 12 days, 13 days, 14 days, 15 days, at least 20 days) before the inhibitor(s) are added.

In the experiments below, the cells were cultured for 4 to 8 days (i.e., day 9) until the cells become sub-confluent, then inhibitor(s) was added, and the cells were cultured for a subsequent 7 to 14 days (i.e., day 10), before the EV collection media (serum-free) was added for 2 days for the collection of EVs. Typically, the EV collection media is free from inhibitors.

The cells most typically begin as a primary keratinocytes. As used herein, “primary keratinocytes” are keratinocytes isolated from tissue and grown in culture, but are not immortalized. In some embodiments, the primary keratinocytes are obtained by a tissue biopsy. In some examples, the tissue biopsy is taken from the skin (e.g., the cutaneous and/or mucosal squamous epithelium). In some embodiments, the primary keratinocyte is a foreskin keratinocyte, a vaginal keratinocyte, a cervical keratinocyte, an oral keratinocyte or a cutaneous keratinocyte.

Typically, primary keratinocytes cultured according to the disclosed methods are considered to be, and can be referred to as, long-term cultured or reprogrammed keratinocytes. Cells treated according to the disclosed methods can exhibit characteristics typical of normal primary keratinocytes, including having a normal karyotype and an intact DNA damage response. In addition, primary keratinocytes long-term cultured or reprogrammed by exposure to a ROCK inhibitor can retain the capacity to differentiate into stratified epithelium upon removal of the ROCK inhibitor.

Thus, in some embodiments, keratinocytes long-term cultured or reprogrammed using a ROCK inhibitor are functionally equivalent or improved compared to normal cells. In some embodiments they have a normal karyotype, an intact DNA damage response, and/or are able to form a stratified epithelium in organotypic culture. In some embodiments, the long-term cultured or reprogrammed keratinocytes exhibit upregulated telomerase mRNA levels and have telomeres that are shortened, but remain at a stable length. Myc mRNA levels may also be increased in ROCK inhibitor long-term cultured or reprogrammed keratinocytes.

In some embodiments, the primary keratinocytes are cultured in the presence of a ROCK inhibitor and/or other inhibitor(s) optionally for a period of time sufficient to allow long-term cultured or reprogramming of the primary keratinocytes, and are further cultured in the absence of the ROCK inhibitor and/or the other inhibitor(s).

In some embodiments, the cultured keratinocytes can differentiate to form the organotypic tissue equivalent. In some embodiment, the organotypic tissue equivalents include primary keratinocytes that have been cultured in the presence of a ROCK inhibitor to increase proliferation of these cells, but the cells are not yet immortalized. Thus, also provided are organotypic tissue equivalents having primary keratinocytes that have been cultured in the presence of a ROCK inhibitor for a period of time sufficient to increase proliferation and/or reprograming of the primary keratinocytes.

Typically the keratinocytes are cultured in the presence of at least a ROCK inhibitor. Rho-associated kinase (also known as and/or referred to herein as ROCK, Rock, Rho-associated coiled-coil kinase, and Rho kinase, includes ROCK1 (also called ROKβ or p160ROCK) and ROCK2 (also called ROKa). ROCK proteins are serine-threonine kinases that interact with Rho GTPases.

Treatment of primary keratinocytes with a ROCK inhibitor can lead to immortalization of these cells. See, e.g., U.S. Published Application No. 2011/0243903, which is specifically incorporated by reference herein in its entirety. In some embodiments, the disclosed method do not immortalize the primary keratinocytes, e.g., as describe in U.S. Published Application No. 2011/0243903.

### A. ROCK Inhibitors

A ROCK inhibitor is a protein, nucleic acid, small molecule, antibody or other agent that reduces or prevents expression of ROCK or down-regulates ROCK activity, such as its kinase activity. Thus, ROCK inhibitors include, but are not limited to, small molecules, antibodies, antisense compounds and negative regulators of ROCK. ROCK inhibitors include inhibitors of ROCK-1, ROCK-2 or both.

The ROCK inhibitor can also be a negative regulator of ROCK, such as, but not limited to small GTP-binding proteins such as Gem, RhoE and Rad. In other examples, the ROCK inhibitor is an antibody that specifically binds ROCK1 or ROCK2 or both isoforms.

In other examples, the ROCK inhibitor is an antisense compound. Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites.

Antisense compounds include, but are not limited to, antisense oligonucleotides, siRNA, miRNA, shRNA and ribozymes. Antisense compounds can specifically target ROCK nucleic acids.

Each of the above-described antisense compounds provides sequence-specific target gene regulation. This sequence-specificity makes antisense compounds effective tools for the selective modulation of a target nucleic acid of interest. In some embodiments, the target nucleic acid is human ROCK1 (e.g., Genbank Accession No. NM_005406) and/or human ROCK2 (Genbank Accession No. NM_004850). However, other known ROCK sequences can be used to design antisense compounds. Methods of designing, preparing and using antisense compounds that specifically target ROCK are within the abilities of one of skill in the art. Examples of ROCK antisense oligonucleotides are described in U.S. Patent Application No. 2004/0115641.

Antisense compounds specifically targeting ROCK1 or ROCK2 can be prepared by designing compounds that are complementary to a ROCK1 or ROCK2 nucleotide sequence. Antisense compounds targeting ROCK1 or ROCK2 need not be 100% complementary to ROCK1 or ROCK2 to specifically hybridize and regulate expression of the target gene. For example, the antisense compound, or antisense strand of the compound if a double-stranded compound, can be at least 75%, at least 80%, at least 85%, at least 90%, at least 95%, at least 99% or 100% complementary to the selected ROCK1 or ROCK2 nucleic acid sequence. Methods of screening antisense compounds for specificity are well known in the art (see, for example, U.S. Patent Application No. 2003/0228689). Antisense compounds can contain one or more modifications to enhance nuclease resistance and/or increase activity of the compound. Modified antisense compounds include those comprising modified internucleoside linkages, modified sugar moieties and/or modified nucleosides.

Preferably, the ROCK inhibitor is a small molecule. Exemplary small molecule ROCK inhibitors include Y-27632 (U.S. Pat. No. 4,997,834, which is specifically incorporated by reference herein in its entirety) and fasudil (also known as HA 1077; Asano et al., J. Pharmacol. Exp. Ther. 241:1033-1040, 1987, which is specifically incorporated by reference herein in its entirety). These inhibitors bind to the kinase domain to inhibit ROCK enzymatic activity. Other small molecules reported to specifically inhibit ROCK include H-1152 ((S)-(+)-2-Methyl-1-[(4-methyl-5-isoquinolinyl) sulfonyl] homopiperazine, Ikenoya et al., J. Neurochem. 81:9, 2002; Sasaki et al., Pharmacol. Ther. 93:225, 2002); N-(4-Pyridyl)-N′-(2,4,6-trichlorophenyl) urea (Takami et al., Bioorg. Med. Chem. 12:2115, 2004); and 3-(4-Pyridyl)-1H-indole (Yarrow et al., Chem. Biol. 12:385, 2005), GSK269962A (Axon medchem), and Fasudil hydrochloride (Tocris Bioscience).

Additional small molecule Rho kinase inhibitors include those described in PCT Publication Nos. WO 03/059913, WO 03/064397, WO 05/003101, WO 04/112719, WO 03/062225 and WO 03/062227; U.S. Pat. Nos. 7,217,722 and 7,199,147; and U.S. Patent Application Publication Nos. 2003/0220357, 2006/0241127, 2005/0182040 and 2005/0197328, each of which is specifically incorporated by reference herein in its entirety.

In another embodiment, the ROCK inhibitor is a negative regulator of ROCK activity. Negative regulators of ROCK activation include small GTP-binding proteins such as Gem, RhoE, and Rad, which can attenuate ROCK activity. Auto-inhibitory activity of ROCK has also been demonstrated upon interaction of the carboxyl terminus with the kinase domain to reduce kinase activity.

In another embodiment, the ROCK inhibitor can be an antibody that specifically binds ROCK1 or ROCK2 or both isoforms. In one example, the antibody specifically binds ROCK1 (e.g., human ROCK1), or ROCK2 (e.g., human ROCK2). By way of example and not limitation, an antibody specific for a ROCK protein can interfere with binding of ROCK to Rho or other binding partners, or the antibody can directly disrupt kinase activity of ROCK.

In a particularly preferred embodiments, the ROCK inhibitor is Y-27632. Also known as (+/−)-trans-N-(4-Pyridyl)-4-(1-aminoethyl)-cyclohexanecarboxamide, Y-27632 is a small molecule inhibitor that selectively inhibits activity of Rho-associated kinase. Y-27632 is disclosed in U.S. Pat. No. 4,997,834 and PCT Publication No. WO 98/06433. In some embodiments, when the ROCK inhibitor is Y-27632, the effective amount of the ROCK inhibitor is about 1 to about 100 μM, or about 5 to about 25 μM, or about 10 μM.

### B. Other Inhibitors

The disclosed culturing methods may include one or more additional inhibitors, for example inhibitors of TGF-beta/Smad signaling. For example, the experimental results below also show that the combination of A83-01 and Y-27632 together appears better for culturing keratinocytes and producing extracellular vesicles than no small molecule inhibitors, but not as well as Y-27632 alone. A83-01 is a potent selective inhibitor of the TGF-BRs ALK4, 5, and 7, which is part of the TGF-β/Smad signaling pathway. Thus, in some embodiments, the cells are cultured with, a protein, nucleic acid, small molecule, antibody or other agent that reduces or prevents expression of a molecule in the TGF-β/Smad signaling pathway or otherwise down-regulates TGF-beta/Smad signaling. Thus, inhibitors of TGF-beta/Smad signaling include, but are not limited to, small molecules, antibodies, antisense compounds and negative regulators of TGF-beta/Smad signaling molecules. Antibodies, antisense compounds and negative regulators can be designed to target TGF-β signaling molecules such as ALK4, 5, and/or 7 according the same strategy discussed above with respect to ROCK inhibitor.

Exemplary small molecule inhibitors of TGF-β/Smad signaling include, but are not limited to, A83-01, SB431542, LDN-193189, Galunisertib (LY2157299), LY2109761, SB525334, SB505124, GW788388, LY364947, RepSox (E-616452), LDN-193189 2HCI, K02288, BIBF-0775, TP0427736 HCl, LDN-214117, SD-208, Vactosertib (TEW-7197), ML347, LDN-212854, DMH1, Dorsomorphin (Compound C), 2HCI, Pirfenidone (S-7701), Sulfasalazine (NSC 667219), AUDA, PD 169316, TA-02, ITD-1, LY 3200882, Alantolactone, Halofuginone, SIS3 HCl, Dorsomorphin (Compound C), and Hesperetin.

Other examples include, but are not limited to, 2-(5-benzo[1,3]dioxole-4-yl-2-tert-butyl-1H-imidazol-4-yl)-6-methylpyridine, 3-(6-methylpyridine-2-yl)-4-(4-quinolyl)-1-phenylthiocarbamoyl-1H-pyrazole (A-83-01), 2-(5-chloro-2-fluorophenyl) pteridine-4-yl)pyridine-4-ylamine (SD-208), 3-(pyridine-2-yl)-4-(4-quinonyl)]-1H-pyrazole, 2-(3-(6-methylpyridine-2-yl)-1H-pyrazole-4-yl)-1,5-naphthyridine (all from Merck) and SB431542 (Sigma Aldrich). A preferred example includes A-83-01. Typically, for example, the inhibitor A83-01 is used concentration of about 1 to about 10 μM, or about 0.1 to about 10 μM, or about 0.5 μM.

Inhibitors are also described in WO 2020/080550, WO 2017/119512, U.S. Pat. No. 10,961,507, and U.S. Ser. No. 17/285,038, each of which is specifically incorporated by reference herein in its entirety.

### III. Extracellular Vesicle

Cell-free compositions including extracellular vesicles (EVs) and methods of use thereof are provided. The EVs can be part of a heterogeneous mixture of factors such as conditioned media, or a fraction isolated therefrom. In other embodiments, EVs, or one or more subtypes thereof, are isolated or otherwise collected from conditioned media. The EVs, or one or more subtypes thereof, can be suspended in a pharmaceutically acceptable composition, such as a carrier or matrix or depot, prior to administration to the subject.

### A. Extracellular Vesicles

The disclosed compositions typically are or include extracellular vesicles derived from primary cultured keratinocytes, or an isolated or fractionated subtype or other cell type derived thereof. Extracellular vesicles are lipid bilayer-delimited particles that are naturally released from a cell and, unlike a cell, cannot replicate. EVs range in diameter from near the size of the smallest physically possible unilamellar liposome (around 20-30 nanometers) to as large as 10 microns or more, although the vast majority of EVs are smaller than 200 nm.

Diverse EV subtypes have been proposed including ectosomes, microvesicles (MV), microparticles, exosomes, oncosomes, apoptotic bodies (AB), tunneling nanotubes (TNT), and more (Yáñez-Mó, et al., J Extracell Vesicles. 4:27066 (2015) doi: 10.3402/jev.v4.27066. PMC 4433489). These EV subtypes have been defined by various, often overlapping, definitions, based mostly on biogenesis (cell pathway, cell or tissue identity, condition of origin) (Théry, et al., J Extracell Vesicles. 7 (1): 1535750 (2018). doi: 10.1080/20013078.2018.1535750, which is specifically incorporated by reference herein in its entirety). However, EV subtypes may also be defined by size, constituent molecules, function, or method of separation. As discussed in Théry, et al., subtypes of EVs may be defined by:
a) physical characteristics of EVs, such as size (“small EVs” (SEVs) and “medium/large EVs” (m/1EVs), with ranges defined, for instance, respectively, <100 nm or <200 nm [small], or >200 nm [large and/or medium]) or density (low, middle, high, with each range defined);
b) biochemical composition (CD63+/CD81+-EVs, Annexin A5-stained EVs, etc.); or
c) descriptions of conditions or cell of origin (podocyte EVs, hypoxic EVs, large oncosomes, apoptotic bodies).

Thus, in some embodiments, the composition is or includes one or more EV subtypes defined according (a), (b), or (c) as discussed above.

In some embodiments, the vesicles are or include exosomes, which may also be referred as, or include, “small EVs”, “sEVs”, etc. Exosomes possess the surface proteins that promote endocytosis and they have the potential to deliver macromolecules. Also, if the exosomes are obtained from the same individual as they are delivered to, the exosomes will be immunotolerant.

Exosomes are vesicles with the size of 30-150 nm, often 40-100 nm, and are observed in most cell types. Exosomes are often similar to MVs with an important difference: instead of originating directly from the plasma membrane, they are generated by inward budding into multivesicular bodies (MVBs). The formation of exosomes includes three different stages: (1) the formation of endocytic vesicles from plasma membrane, (2) the inward budding of the endosomal vesicle membrane resulting in MVBs that consist of intraluminal vesicles (ILVs), and (3) the fusion of these MVBs with the plasma membrane, which releases the vesicular contents, known as exosomes.

Exosomes have a lipid bilayer with an average thickness of ˜5 nm (see e.g., Li, Theranostics, 7 (3): 789-804 (2017) doi: 10.7150/thno.18133). The lipid components of exosomes include ceramide (sometimes used to differentiate exosomes from lysosomes), cholesterol, sphingolipids, and phosphoglycerides with long and saturated fatty-acyl chains. The outer surface of exosomes is typically rich in saccharide chains, such as mannose, polylactosamine, alpha-2,6 sialic acid, and N-linked glycans.

Many exosomes contain proteins such as platelet derived growth factor receptor, lactadherin, transmembrane proteins and lysosome associated membrane protein-2B, membrane transport and fusion proteins like annexins, flotillins, GTPases, heat shock proteins, tetraspanins, proteins involved in multivesicular body biogenesis, as well as lipid-related proteins and phospholipases. These characteristic proteins therefore serve as good biomarkers for the isolation and quantification of exosomes. Another key cargo that exosomes can carry is nucleic acids including deoxynucleic acids (DNA), coding and non-coding ribonucleic acid (RNA) like messenger RNA (mRNA) and microRNA (miRNA).

In some embodiments, the vesicles include or are one or more alternative extracellular vesicles, such as ABs, MVs, TNTs, or others discussed herein or elsewhere.

ABs are heterogenous in size and originate from the plasma membrane. They can be released from all cell types and are about 1-5 μm in size.

MVs with the size of 20 nm-1 μm are formed due to blebbing with incorporation of cytosolic proteins. In contrast to ABs, the shape of MVs is homogenous. They originate from the plasma membrane and are observed in most cell types.

TNTs are thin (e.g., 50-700 nm) and up to 100 μm long actin containing tubes formed from the plasma membrane.

In some embodiments, the EVs are between about 20 nm and about 500 nm. In some embodiments, the EVs are between about 20 nm and about 250 nm or 200 nm or 150 nm or 100 nm.

### B. Methods of Making Extracellular Vesicles

### 1. Sources of Cells for Making Extracellular Vesicles

As used herein, EVs, including AB, MV, exosomes, and TNT typically refer to lipid vesicles formed by cells or tissue. Generally, EVs can be isolated from tissue, cells, and fluid directly from a subject, including cultured and uncultured tissue, cells, or fluids, and fluid derived or conditioned by cultured cells (e.g., conditioned media). For example, exosomes are present in physiological fluids such as plasma, lymph liquid, malignant pleural effusion, amniotic liquid, breast milk, semen, saliva and urine, and are secreted into the media of cultured cells.

The disclosed EVs are typically formed from cultured primary keratinocytes as disclosed herein.

Methods of isolating extracellular vesicles from tissue, cells, and fluid directly from a subject, including cultured and uncultured tissue, cells, or fluids, and fluid derived or conditioned by cultured cells (e.g., conditioned media) are known in the art. See, for example, Li, Thernaostics, 7 (3): 789-804 (2017) doi: 10.7150/thno.18133, Ha, et al., Acta Pharmaceutica Sinica B, 6 (4): 287-296 (2016) doi: 10.1016/j.apsb.2016.02.001, Skotland, et al., Progress in Lipid Research, 66:30-41 (2017) doi: 10.1016/j.plipres.2017.03.001, Phinney and Pittenger, Stem Cells, 35:851-858 (2017) doi: 10.1002/stem.2575, each of which is specifically incorporated by reference, and describes isolating extracellular vesicles, particularly exosomes.

The disclosed EVs are typically collected from cultured primary cells or a subsequent cell type derived therefrom. In some embodiments, the vesicles are isolated from primary cells isolated from the subject to be treated. An advantage of utilizing EVs that can be isolated from natural sources includes avoidance of immunogenicity that can be associated with artificially produced lipid vesicles.

The EVs can also be collected from cell lines or tissue. The disclosed EVs are most typically collected from keratinocytes cultured as described herein. In some embodiments, the media of cultured keratinocytes is changed prior to collect of the EVs. Such media can be described as collection media, and may be the same or different than the culture media. The collection media can, but need not, include the one or more inhibitors used to culture the cells.

### 2. Methods of Collecting Extracellular Vesicles

Extracellular vesicles, including exosomes, can be isolated using differential centrifugation, flotation density gradient centrifugation, filtration, high performance liquid chromatography, and immunoaffinity-capture.

For example, one of the most common isolation technique for isolating exosomes from cell culture is differential centrifugation, whereby large particles and cell debris in the culture medium are separated using centrifugal force between 200-100,000×g and the exosomes are separated from supernatant by the sedimenting exosomes at about 100,000×g. Purity can be improved, however, by centrifuging the samples using flotation density gradient centrifugation with sucrose or Optiprep. Tangential flow filtration combined with deuterium/sucrose-based density gradient ultracentrifugation was employed to isolate therapeutic exosomes for clinical trials.

Ultrafiltration and high performance liquid chromatography (HPLC) are additional methods of isolating EVs based on their size differences. EVs prepared by HPLC are highly purified.

Hydrostatic filtration dialysis has been used for isolating extracellular vesicles from urine.

Other common techniques for EV collection involve positive and/or negative selection using affinity-based methodology. Antibodies can be immobilized in different media conditions and combined with magnetic beads, chromatographic matrix, plates, and microfluidic devices for separation. For example, antibodies against exosome-associated antigens-such as cluster of differentiation (CD) molecules CD63, CD81, CD82, CD9, epithelial cell adhesion molecule (EpCAM), and Ras-related protein (Rab5)—can be used for affinity-based separation of exosomes. Non-exosome vesicles that carry these or different antigens can also be isolated in a similar way.

Microfluidics-based devices have also been used to rapidly and efficiently isolate EVs such as exosomes, tapping on both the physical and biochemical properties of exosomes at microscales. In addition to size, density, and immunoaffinity, sorting mechanisms such as acoustic, electrophoretic and electromagnetic manipulations can be implemented.

Methods of characterizing EVs including exosomes are also known in the art. Exosomes can be characterized based on their size, protein content, and lipid content. Exosomes are sphere-shaped structures with sizes between 40-100 nm and are much smaller compared to other systems, such as a microvesicle, which has a size range from 100-500 nm. Several methods can be used to characterize EVs, including flow cytometry, nanoparticle tracking analysis, dynamic light scattering, western blot, mass spectrometry, and microscopy techniques. EVs can also be characterized and marked based on their protein compositions. For example, integrins and tetraspanins are two of the most abundant proteins found in exosomes. Other protein markers include TSG101, ALG-2 interacting protein X (ALIX), flotillin 1, and cell adhesion molecules. Similar to proteins, lipids are major components of EVs and can be utilized to characterize them.

### C. Pharmaceutical Compositions

Pharmaceutical compositions including EVs and/or cells are also provided. Pharmaceutical compositions can be administered parenterally (intramuscular (IM), intraperitoneal (IP), intravenous (IV), subcutaneous injection (SubQ), subdermal), transdermally (either passively or using iontophoresis or electroporation), or by any other suitable means, and can be formulated in dosage forms appropriate for each route of administration.

In some embodiments, the compositions are administered systemically, for example, by intravenous or intraperitoneal administration, in an amount effective for delivery of the compositions to targeted cells.

In preferred embodiments, the compositions are administered locally, for example, by injection directly into, or adjacent to, a site to be treated. Typically, local injection causes an increased localized concentration of the compositions which is greater than that which can be achieved by systemic administration.

In some embodiments, the compositions are delivered locally to the appropriate cells by using a catheter or syringe. Other means of delivering such compositions locally to cells include using infusion pumps (for example, from Alza Corporation, Palo Alto, Calif.) or incorporating the compositions into polymeric implants (see, for example, P. Johnson and J. G. Lloyd-Jones, eds., Drug Delivery Systems: Fundamentals and Techniques (Chichester, England: Ellis Horwood Ltd., 1988 ISBN-10:0895735806), which can affect a sustained release of the material to the immediate area of the implant.

The EV compositions can be provided to the cells either directly, such as by contacting it to or with the cells, or indirectly, such as through the action of any biological process. For example, the vesicles can be formulated in a physiologically acceptable carrier and injected into a tissue or fluid surrounding the cells.

Exemplary dosage for in vivo methods are discussed in the experiments below. As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired.

Generally, for local injection or infusion, dosage may be lower. Generally, the total amount of the active agent administered to an individual using the disclosed vesicles can be less than the amount of unassociated active agent that must be administered for the same desired or intended effect and/or may exhibit reduced toxicity.

In a preferred embodiment the compositions are administered in an aqueous solution, by parenteral injection such as intramuscular, intraperitoneal, intravenous, subcutaneous, subdermal, etc.

The formulation can be in the form of a suspension or emulsion. In general, pharmaceutical compositions are provided including effective amounts of one or more active agents optionally include pharmaceutically acceptable diluents, preservatives, solubilizers, emulsifiers, adjuvants and/or carriers. Such compositions can include diluents, sterile water, buffered saline of various buffer content (e.g., Tris-HCl, acetate, phosphate) at various pHs and ionic strengths; and optionally, additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacterium retaining filter, by incorporating sterilizing agents into the compositions, by irradiating the compositions, or by heating the compositions.

Transdermal formulations may also be prepared. These will typically be ointments, lotions, sprays, or patches, all of which can be prepared using standard technology. Transdermal formulations can include penetration enhancers. Chemical enhancers and physical methods including electroporation and microneedles can work in conjunction with this method. Typically the penetration enhancer(s) are selected such that it/they do not disrupt and/or eliminate the biological activity of the EVs.

### D. Methods of Use

Methods of using the disclosed compositions are also provided. In some embodiments, the methods include contacting cells, or administering to a subject in need thereof, an effective amount of a composition including extracellular vesicles.

Resident skin cells such as keratinocytes, fibroblasts, melanocytes, and inflammatory cells can secrete different types of EVs depending on their biological state (Nasiri, et al., “Shedding light on the role of keratinocyte-derived extracellular vesicles on skin-homing cells,” Stem Cell Research & Therapy, volume 11, Article number: 421 (2020), which is specifically incorporated by reference herein in its entirety). These vesicles can influence the physiological properties and pathological processes of skin, such as pigmentation, cutaneous immunity, and wound healing. Since keratinocytes constitute the majority of skin cells, secreted EVs from these cells may alter the pathophysiological behavior of other skin cells. For example, keratinocyte EVs have been shown to harbor a variety of biomolecules including DNA, miRNA, mRNA, and proteins. They are believed to facilitate cross-talk between keratinocytes and melanocytes, keratinocytes and immune cells, modulate cell proliferation, migration, and angiogenesis during homeostasis and wound healing. It is thus believed that keratinocyte EVs can be used for nutraceutical and therapeutic approaches. For example, the physiological function of keratinocyte-derived exosomes in the regulation of melanocyte proteins is also well established, and may offer a therapeutic approach for hypo- and hyperpigmentation disorders. Furthermore, keratinocyte-derived exosomes can function as intercellular transmitters and immune modulators through interaction with APCs, which may provide a therapeutic approach through the reduction of immune responses.

In some embodiments, exosome/EVs produced according to the disclosed methods can increase the expression of Type I collagen (COL1A1) and/or elastin in cell contacted with the exosomes. Such cells can include, but are not limited to, fibroblasts. Collagen and elastin are the main fibers that form the extracellular matrix. See, e.g., Mehta-Ambalal, J Cutan Aesthet Surg. 2016 July-September; 9 (3): 145-151. doi: 10.4103/0974-2077.191645. Both are formed by fibroblasts. Collagen is responsible for tensile strength and elastin provides elasticity to the skin. Production and density of both decreases as a function of age, and results in sagging and wrinkling. Wounding alters the amount and quality of these fibers. Thus, exosome made according to the disclosed methods can be used to manage aesthetic conditions such as cutaneous ageing and scarring.

Additionally, or alternatively, specific, additional therapeutic molecules like genetics materials, proteins, or even inhibitor agents can be engineered into EVs and, for example, delivered to the target abnormal cells, i.e., fibroblasts, melanocytes, or inflammatory cells, in order to improve their biological activity for the treatment of skin disorders such as pigmentation abnormalities, autoimmune disease like psoriasis, chronic wound, etc, methods of loading drug into pre-formed vesicles including exosomes are known in the art and reviewed in Ha, et al., Acta Pharmaceutica Sinica B, 6 (4): 287-296 (2016) doi: 10.1016/j.apsb.2016.02.001, and discussed in Yang, et al., J Control Release, 243:160-171 (2016). doi: 10.1016/j.jconrel.2016.10.008, each of which are specifically incorporated by reference.

Briefly, small molecules have been loaded by mixing and incubation and through complexation with, for example, surface elements. Proteins and peptides have been loaded by incubation, with or without a permeabilizer such as saponin, through freeze-thaw cycling, sonication, and extrusion procedures. Nucleic acids have been loaded by chemical transfection and electroporation. See also Table 2 of Ha, et al., Acta Pharmaceutica Sinica B, 6 (4): 287-296 (2016) doi: 10.1016/j.apsb.2016.02.001, and the references cited therein.

Thus, in some embodiments, the disclosed compositions are contacted with cells or administered to a subject in need thereof in an effective amount to have a biochemical or physiological effect on one or more cell types of the skin (e.g., keratinocytes, fibroblasts, melanocytes, inflammatory cells, etc.). In some embodiments, the disclosed compositions are administered to a subject in need thereof in an effective amount to have a such as nutraceutical or therapeutic effect. In some embodiments, the compositions are topically administered, e.g., by contact with the skin of the subject. Exemplary, non-limiting diseases include skin disorders such as pigmentation abnormalities, autoimmune disease like psoriasis, chronic wounds, atopic dermatitis, etc., and others mentioned herein and elsewhere.

In some embodiments, the compositions are used to treat or prevent skin, irritation, stress, allergies, infection and/or heat/sweating of the skin. For example, the experiments below show that keratinocyte EVs prepared according to the disclosed methods inhibited TSLP, IL-25, and IL-33, factors that are highly related to induction of pathogenesis of atopic dermatitis. Atopic dermatitis (as known as eczema) is a condition characterized by red and itchy skin. It is common in children but can occur at any age. Atopic dermatitis is long lasting (chronic) and tends to flare periodically, and may be accompanied by asthma or hay fever. Atopic dermatitis symptoms vary widely from person to person and can include: dry skin; itching, which may be severe, especially at night; red to brownish-gray patches, especially on the hands, feet, ankles, wrists, neck, upper chest, eyelids, inside the bend of the elbows and knees, and in infants, the face and scalp; small, raised bumps, which may leak fluid and crust over when scratched; thickened, cracked, scaly skin; and raw, sensitive, swollen skin from scratching.

In some embodiments, the disclosed compositions (e.g., EV's prepared by the culturing methods disclosed and/or compositions formed therefrom) are more effective than counterpart compositions prepared according to a traditional (e.g., non-long-term, non-reprogrammed method). In some embodiments, the traditional method does not include culturing the keratinocytes with a ROCK inhibitor and/or an inhibitor of TGFβ signaling. Thus, in some embodiments, the traditional culturing method is free from culturing the keratinocytes with a ROCK inhibitor and/or an inhibitor of TGFβ signaling.

For example, the experiments below show that the inhibitory effect of EV's prepared according to the long-term culturing methods disclosed herein are much stronger than EV's prepared according to traditional keratinocyte culturing methods. Thus, in some embodiments, the skin disease or disorder to be treated is atopic dermatitis. In some embodiments, EV's prepared by the disclosed methodology reduce or prevent one or more symptoms or biochemical or physiological indicators of atopic dermatitis. Biochemical and physiological indicators can include, but are not limited to, thymic stromal lymphopoietin (TSLP), Th2, eosinophil-recruiting chemokines, inflammatory cytokines such as IL-33 and IL-25, and combinations thereof. Thus, in some embodiments, EV's prepared by the disclosed methodology reduce or prevent one or more symptoms or biochemical or physiological indicators of atopic dermatitis to a greater degree than EV's prepared according to a traditional (e.g., non-long-term) culturing method.

In some embodiments, the EVs are administered as part of a heterogeneous mixture of factors (e.g., conditioned media, or a fraction isolated therefrom). In some embodiments, EVs or more of more subtypes thereof are isolated or otherwise collected from conditioned media. The EVs or one or more subtypes thereof can be suspended in pharmaceutically acceptable composition, such as a carrier or matrix or depot, prior to administration to the subject.

EVs may possess the versatility and capacity to interact with multiple cell types immediately and in remote areas to regulate cellular responses (Zhang et al., Cell Prolif., 49:3-13 (2016)). Thus, although regional or local administration to the site of interest or a site adjacent thereto is preferred, systemic administration is also contemplated.

The frequency of administration of a method of treatment can be, for example, one, two, three, four or more times daily, weekly, every two weeks, or monthly. In some embodiments, the composition is administered to a subject once every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. In some embodiments, the frequency of administration is once, twice or three times weekly, or is once, twice or three times every two weeks, or is once, twice or three times every four weeks. In some embodiments, the composition is administered to a subject 1-3 times, preferably 2 times, a week.

In some embodiments, the effect of the disclosed compositions and methods on a subject is compared to a control. For example, the effect of the composition on a particular symptom, pharmacologic, or physiologic indicator (including those mentioned above and elsewhere herein) can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art, such as one of those discussed herein.

### E. Kits

Dosage units including the disclosed compositions, for example, in a pharmaceutically acceptable carrier for shipping and storage and/or administration are also disclosed. Components of the kit may be packaged individually and can be sterile. In some embodiments, a pharmaceutically acceptable carrier containing an effective amount of the composition is shipped and stored in a sterile vial. The sterile vial may contain enough composition for one or more doses. The composition may be shipped and stored in a volume suitable for administration, or may be provided in a concentration that is diluted prior to administration. In another embodiment, a pharmaceutically acceptable carrier containing drug can be shipped and stored in a syringe.

Kits containing syringes of various capacities or vessels with deformable sides (e.g., plastic vessels or plastic-sided vessels) that can be squeezed to force a liquid composition out of an orifice are provided. The size and design of the syringe will depend on the route of administration. Any of the kits can include instructions for use.

The invention can be further understood by the following numbered paragraphs:
1. A method of making extracellular vesicles (EVs) comprising culturing keratinocytes in culture media comprising a ROCK inhibitor, and harvesting EVs secreted by the keratinocytes.
2. The method of paragraph 1, wherein the ROCK inhibitor is Y-27632.
3. The method of paragraph 2, wherein the Y-27632 is in a concentration of about 1 μM to about 100 μM, or about 5 μM to about 25 μM, or about 10 μM.
4. The method of any one of paragraphs 1-3, wherein the cells are cultured with an inhibitor of TGFβ signaling.
5. The method of paragraph 4, wherein the inhibitor of TGFβ signaling is A83-01.
6. The method of paragraph 5, wherein the A83-01 is in concentration of about 1 μM to about 10 μM, or about 0.1 μM to about 10 μM, or about 0.5 μM.
7. The method of any one of paragraphs 1-6, wherein proliferation and/or secretion of EVs is increased in the presence of the ROCK inhibitor compared to its absence.
8. The method of any one of paragraphs 1-7, wherein the cells are cultured in the ROCK inhibitor and optionally inhibitor of TGFβ signaling for at least 5, 6, 7, 8, 9, 10, 11, 12, 13, 14, 15, 16, 17, 18, 19, or 20 days; or about 5 days to about 25 days, or any subrange or integer number of days therebetween, optionally for about 7 days to about 22 days, about 5 days to about 25 days, or about 10 days to about 20 days, or about 12 days to about 17 days; or about 13, 14, or 15 days.
9. The method of any one of paragraphs 1-8, wherein the EVs comprise or consists of exosomes.
10. Extracellular vesicles (EVs) made according to the method of any one of paragraphs 1-9.
11. A pharmaceutical composition comprising an effective amount of the EVs of paragraph 10.
12. A therapeutic or non-therapeutic method of treating a subject, comprising administering the subject the pharmaceutical composition of paragraph 11.
13. The method of paragraph 12, wherein the subject has a skin disease or disorder or injury.
14. A therapeutic or non-therapeutic method of improving the skin of a subject in need thereof comprising administering the subject the pharmaceutical composition of paragraph 11.
15. A therapeutic or non-therapeutic method of reducing or preventing cutaneous ageing and scarring of the skin comprising administering the subject the pharmaceutical composition of paragraph 11.
16. The method of any one of paragraphs 12-15, wherein the method comprises contacting skin and/or cells thereof with the pharmaceutical composition.
17. The method of any one of paragraphs 12-16, wherein the pharmaceutical composition increases expression of Type I collagen (COL1A1) in cells of the subject
18. The method of any of paragraphs 12-17, wherein the pharmaceutical composition increases expression of the Elastin in cells of the subject.
19. The method of any one of paragraphs 16-18, wherein the cells are fibroblasts.
20. The method of paragraph 19, wherein the fibroblasts are dermal fibroblasts.
21. A method of treating atopic dermatitis comprising administering the subject the pharmaceutical composition of paragraph 11.
22. The method of paragraph 21, wherein the method comprises contacting skin and/or cells thereof with the pharmaceutical composition.
23. The method of paragraph 22, wherein the skin of the subject is dry, scaly, raw, sensitive, swollen, red, comprises bumps, or a combination thereof.
24. The method of any one of paragraphs 21-23, wherein the pharmaceutical composition reduces expression of thymic stromal lymphopoietin (TSLP), Th2, eosinophil-recruiting chemokines, inflammatory cytokines such as IL-33 and IL-25, or a combination thereof in cells of the subject
25. The method of paragraph 24, wherein the pharmaceutical composition reduces expression of TSLP, IL-25, IL-33 or a combination thereof in cells of the subject.
26. The method of any one of paragraphs 16-18, wherein the cells are keratinocytes.
27. The method of paragraph 26, wherein the keratinocytes are epidermal keratinocytes.
28. The method of any one of paragraphs 12-27, wherein the EV's are more effective than EV's prepared according to a non-long-term or non-reprogramming keratinocyte culturing method.
29. The method of paragraph 28, wherein the non-long-term or non-reprogramming keratinocyte culturing method is free from culturing the keratinocytes with a ROCK inhibitor.
30. The method of paragraphs 28 or 29, wherein the non-long-term or non-reprogramming keratinocyte culturing method is free from culturing the keratinocytes with an inhibitor of TGFβ signaling.

### EXAMPLES

### Example 1: Rock Inhibitor Enhances Secretion of Extracellular Vesicles During Long Term Culture of Keratinocytes

### Materials and Methods

“Y” refers to Y-27632 (Rock inhibitor)
“A” refers to A83-01 (TGFβ signaling inhibitor)
“KC” refers to keratinocyte cells

Human primary keratinocytes were cultured in EpiLife™ medium with or without Y or Y+A supplementation according to the schedule below, and analyzed for extracellular vesicle secretion. 10 μM Y-27632 (Wako) and 0.5 μM A-83-01 (Wako) was used. Collection media was serum-free EpiLife™ medium without inhibitors. Thus, depending on the experiment, or stage of the experiment, human epidermal keratinocytes were cultured in serum free media such as EpiLife™ Medium, without or without small molecule inhibitor(s), namely, 10 μM Y-27632 (Wako) alone, or 10 μM Y-27632 plus 0.5 μM A-83-01 (Wako).

TABLE 1
Culture and Collection Protocol

| Steps | | Day |
|---|---|---|
| Raise cells → 25 flasks × 3 | | 1 |
| Medium exchange | | 3 |
| Medium exchange | | 7 |
| Cell passage → 10 cm | control: Culture with | 9 |
| dish × 3 | Epilife, culture with Y: | |
| | Epilife + Y, start culture | |
| | with YA: Epilife + Y | |
| Medium exchange | | 12 |
| Medium exchange | | 15 |
| Cell passage Y, YA only | only Y and YA | 17 |
| Medium exchange | Medium was exchanged to | 19 |
| | EVs recovery medium (Epilife). | |
| Collection of cell | Collect each medium after | 21 |
| culture supernatant | 48 hours → Store at 4° C. | |
| | after 0.22 μm filtration | |
| Nanosight analysis | | 26 |

### Results

Long-term culture of keratinocytes was carried out by adding low molecular weight compounds Y and Y+A.

The amount of EVs secreted from normal KC (EpiLife culture only control) was compared with the amount of EVs secreted from KC cultured for a long time with low molecular weight compounds Y and YA.

Results showed that culturing with low molecular weight compound Y increased cell proliferation and EVs secretion. Untreated cells showed slightly advanced cell differentiation. Y treated cells showed good cell growth, and good morphology. YA treated cells were in poor condition and showed poor adhesion.

By culturing with Y, cell proliferation was enhanced as compared with the case of culturing normal cells without the addition of Y. Compared with normal KC-EVs, the amount of EVs secreted increased by culturing with low molecular weight compounds Y and YA. However, the cell morphology was the best for low molecular weight compound Y, and the cell condition was poor in YA culture.

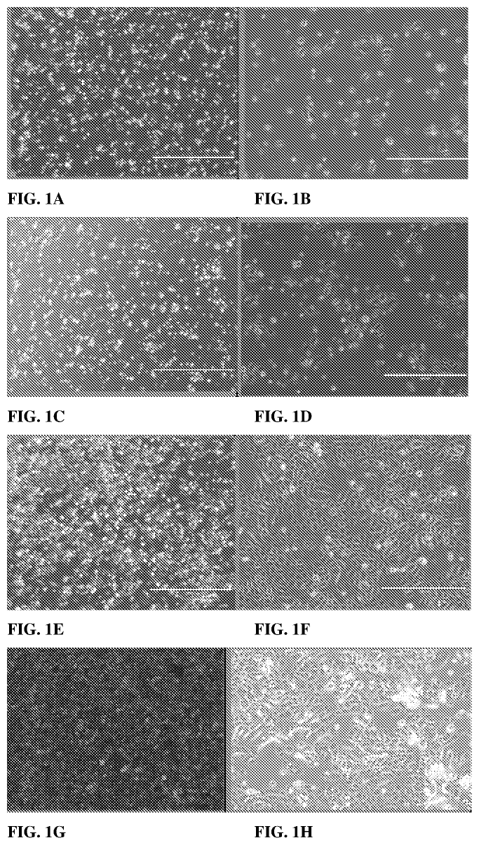

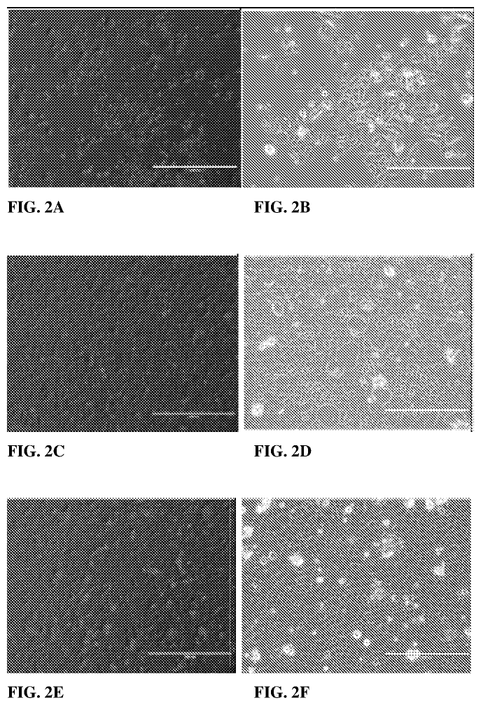

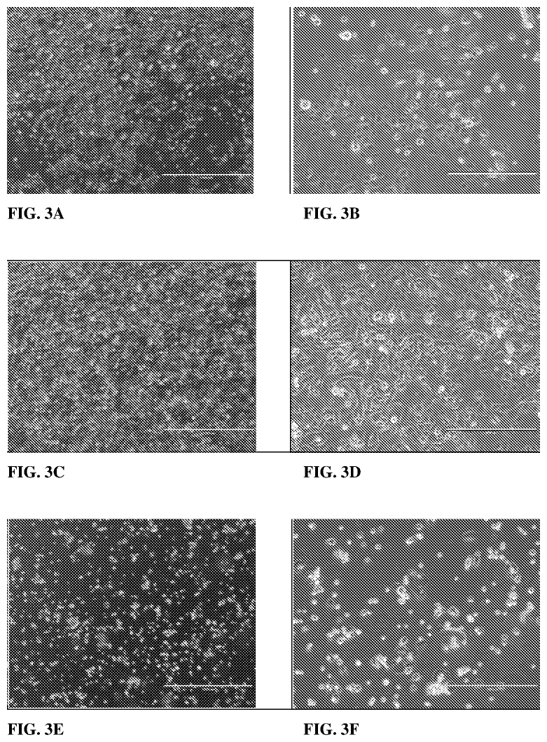

Results are illustrated in Table 2 (below), and FIGS. 1A-5.

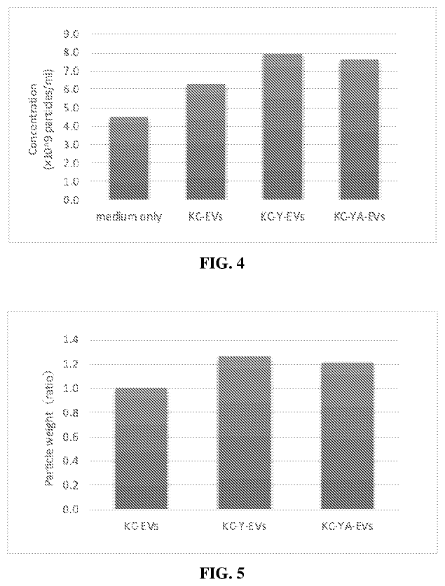

Inhibitor treated-keratinocytes (Y alone or Y+A) secreted a larger number of exosome/EVs compared to original culture of keratinocytes without inhibitors (see e.g., FIGS. 4 & 5). Inhibitor-treated keratinocytes exosomes/EVs showed CD9- and CD63-positive. The nanoparticle tracking system nanosight showed that the particles are around 100 nm in diameter size, which are consistent with the particles being exosomes or small EVs.

TABLE 2
Results of culture keratinocytes with or without Y or Y + A.

| | | | | Amount | | | | | | | |
|---|---|---|---|---|---|---|---|---|---|---|---|
| | | | | of | | | | | | | |
| | | | | recovered | | | | | Concentration | | Particle |
| | | Culture | | medium | | Solution | camera | | (×10\hat{ }9 | | size |
| sample | | medium | passage | (mL) | treatment | dilution | level | detection | particles/ml) | ratio | (nm) |
| 1 | medium | — | — | 15 | 0.22 μm | 2 | 13 | 7 | 4.5 | | 97.5 |
| | only | | | | filtration | | | | | | |
| 2 | KC-EVs | Culture in | p2 | 15 | 0.22 μm | 2 | 13 | 7 | 6.3 | 1.0 | 93.7 |
| | | Epilife | | | filtration | | | | | | |
| 3 | KC-Y-EVs | Culture | p3 | 15 | 0.22 μm | 2 | 13 | 7 | 8.0 | 1.3 | 88.3 |
| | | with | | | filtration | | | | | | |
| | | Epilife + Y | | | | | | | | | |
| 4 | KC-YA- | Culture | p3 | 15 | 0.22 μm | 2 | 13 | 7 | 7.6 | 1.2 | 107.1 |
| | EVs | with | | | filtration | | | | | | |
| | | Epilife + | | | | | | | | | |
| | | YA | | | | | | | | | |

### Example 2: Exosomes from Cultured Keratinocytes Increase Expression of Collagen and Elastin

### Materials and Methods

Exosomes/EVs were harvested from keratinocytes treated with Y (inhibitor) at 10 μM Y-27632 for 14 days and replaced the culture media to and the harvested culture supernatant. The culture supernatant was filtered to remove cell debris and then ultracentrifuge to harvest and purify exosomes/EVs. The purified exosomes/EVs were resuspended in PBS (−) and the number of particles were counted with Nanosight.

Culture of human skin fibroblasts: Human Dermal Fibroblast, Adult, Normal, Cryopreserved <NHDF-c Adult>: C-12302 Funakoshi p2 cultured in HFDM-1 Medium (Funakoshi 2102P05) up to p10 (passage 10 times) (70% confluent. Cell culture) dish 6 cm).

Keratinocyte exosomes/EVs were added to human fibroblasts (cultured in DMEM supplemented with glucose) at 1,000 exosomes per cell.

72 hours later fibroblasts were recovered and mRNA was prepared from cells with Qiagen's RNeasy Mini kit, cDNA was synthesized, and the expression levels of Type I collagen (COL1A1) (ThermoFisher Assay ID: Hs00164004_m1) and elastin (ThermoFisher Assay ID: Dr03073243_g1) were quantified by quantitative PCR with Taqman probe (Catalog number: 4331182).

### Results

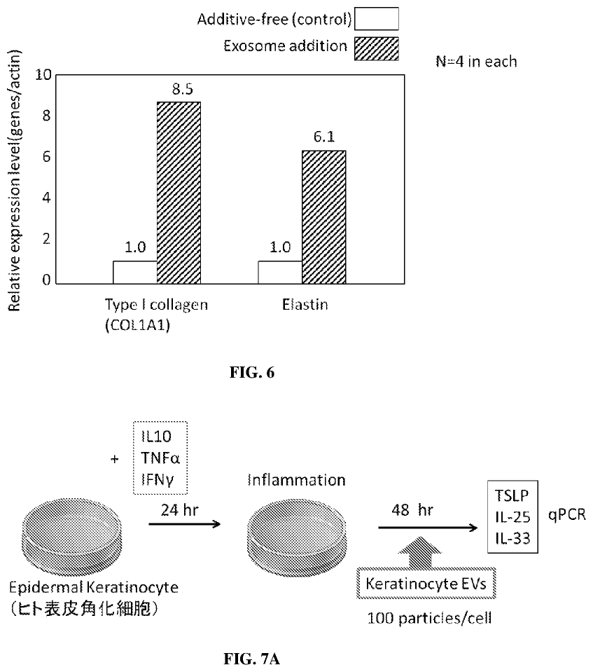

Results are presented in FIG. 6 and show that exosome particles secreted by human keratinocytes treated with low molecular weight compound induce expression of Type I collagen and Elastin genes in human fibroblasts. These results are consistent with a skin-beautifying effect of exosomes derived from keratinocytes treated with low molecular weight compound.

### Example 3: Exosomes from Cultured Keratinocytes Inhibit Factors that Induce Atopic Dermatitis

### Materials and Methods

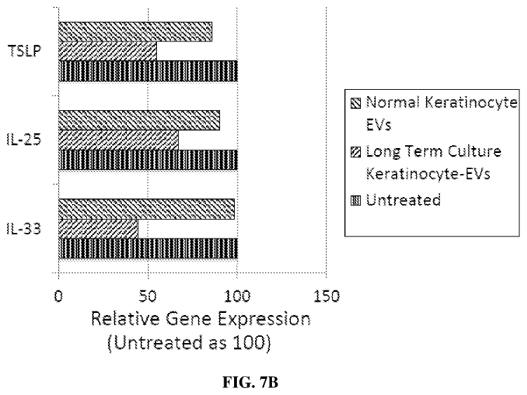

An in vitro model for atopic dermatitis was developed and is illustrated in FIG. 7A. Epidermal keratinocytes were cultured with 10 μM IL-10, TNFα, and IFNγ for 24 hours to induce inflammation.

Long term keratinocytes were prepared by treating keratinocytes with 10 μM Y-27632 for 14 days. Exosomes/EVs were prepared by culturing normal keratinocytes (EpiLife only) and long-term keratinocytes (EpiLife only) for 48 hours and collecting the supernatant. The culture supernatant was filtered to remove cell debris and then ultracentrifuge to harvest and purify exosomes/EVs. The purified exosomes/EVs were resuspended in PBS (−) and the number of particles were counted with Nanosight.

Keratinocyte exosomes/EVs were added to epidermal keratinocytes for 48 hours at a concentration of 100 particles/cell. Gene expression of TSLP, IL-25, and IL-33 were analyzed by qPCR.

### Results

Barrier disruption and keratinocyte injuries that stimulate thymic stromal lymphopoietin (TSLP), Th2, and eosinophil-recruiting chemokines together with IL-33 and IL-25 released from keratinocytes are key players in the pathogenesis of atopic dermatitis. See, e.g., Rerknimitr, et al., Inflamm Regen. 37:14, doi: 10.1186/s41232-017-0044-7 (2017).

An in vitro model was developed and used to test the impact of keratinocyte exosomes/EVs on atopic dermatitis pathogenesis. Results, presented in FIG. 7B, show that keratinocyte exosomes/EVs prepared using long-term culturing significantly inhibited TSLP, IL-25, and IL-33, which are highly related to induction of pathogenesis of atopic dermatitis. The inhibitory effect of keratinocyte exosomes/EVs prepared by long-term culturing was much stronger than that of exosomes/EVs prepared from normal cultured keratinocytes. These results support a conclusion that exosomes/EVs harvested from reprogrammed keratinocytes may be an effective treatment for atopic dermatitis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method of making extracellular vesicles (EVs) comprising culturing keratinocytes in culture media comprising a ROCK inhibitor, and harvesting EVs secreted by the keratinocytes.

2. The method of claim 1, wherein the ROCK inhibitor is Y-27632.

3. The method of claim 2, wherein the Y-27632 is in a concentration of about 1 μM to about 100 μM, or about 5 μM to about 25 μM, or about 10 μM.

4. The method of claim 1, wherein the cells are cultured with an inhibitor of TGFβ signaling.

5. The method of claim 4, wherein the inhibitor of TGFβ signaling is A83-01.

6. The method of claim 5, wherein the A83-01 is in concentration of about 1 μM to about 10 μM, or about 0.1 μM to about 10 μM, or about 0.5 μM.

7. The method of claim 1, wherein proliferation and/or secretion of EVs is increased in the presence of the ROCK inhibitor compared to its absence.

8. The method of claim 1, wherein the cells are cultured in the ROCK inhibitor and optionally inhibitor of TGFβ signaling for at least 5, 6, 7, 8, 9, 10, 11, 12, 13, 14, 15, 16, 17, 18, 19, or 20 days; or about 5 days to about 25 days, or any subrange or integer number of days therebetween, optionally for about 7 days to about 22 days, about 5 days to about 25 days, or about 10 days to about 20 days, or about 12 days to about 17 days; or about 13, 14, or 15 days.

9. The method of claim 1, wherein the EVs comprise or consists of exosomes.

10. Extracellular vesicles (EVs) made according to the method of claim 1.

11. A pharmaceutical composition comprising an effective amount of the EVs of claim 10.

12. A therapeutic or non-therapeutic method of treating a subject, comprising administering the subject the pharmaceutical composition of claim 11.

13. The method of claim 12, wherein the subject has a skin disease or disorder or injury.

14. A therapeutic or non-therapeutic method of improving the skin of a subject in need thereof comprising administering the subject the pharmaceutical composition of claim 11.

15. A therapeutic or non-therapeutic method of reducing or preventing cutaneous ageing and scarring of the skin comprising administering the subject the pharmaceutical composition of claim 11.

16. The method of claim 12, wherein the method comprises contacting skin and/or cells thereof with the pharmaceutical composition.

17. The method of claim 12, wherein the pharmaceutical composition increases expression of Type I collagen (COL1A1) in cells of the subject

18. The method of claim 12, wherein the pharmaceutical composition increases expression of the Elastin in cells of the subject.

19. The method of claim 16, wherein the cells are fibroblasts.

20. The method of claim 19, wherein the fibroblasts are dermal fibroblasts.

21. A method of treating atopic dermatitis comprising administering the subject the pharmaceutical composition of claim 11.

22. The method of claim 21, wherein the method comprises contacting skin and/or cells thereof with the pharmaceutical composition.

23. The method of claim 22, wherein the skin of the subject is dry, scaly, raw, sensitive, swollen, red, comprises bumps, or a combination thereof.

24. The method of claim 21, wherein the pharmaceutical composition reduces expression of thymic stromal lymphopoietin (TSLP), Th2, eosinophil-recruiting chemokines, inflammatory cytokines such as IL-33 and IL-25, or a combination thereof in cells of the subject

25. The method of claim 24, wherein the pharmaceutical composition reduces expression of TSLP, IL-25, IL-33 or a combination thereof in cells of the subject.

26. The method of claim 16, wherein the cells are keratinocytes.

27. The method of claim 26, wherein the keratinocytes are epidermal keratinocytes.

28. The method of claim 12, wherein the EV's are more effective than EV's prepared according to a non-long-term or non-reprogramming keratinocyte culturing method.

29. The method of claim 28, wherein the non-long-term or non-reprogramming keratinocyte culturing method is free from culturing the keratinocytes with a ROCK inhibitor.

30. The method of claim 28, wherein the non-long-term or non-reprogramming keratinocyte culturing method is free from culturing the keratinocytes with an inhibitor of TGFβ signaling.
